(19) United States
(12) Patent Application Publication
(10) Pub. No.: US 2021/0321973 A1
(43) Pub. Date: Oct. 21, 2021

(54) ELECTRONIC ACOUSTIC COLLECTION SYSTEM

(71) Applicant: StratoScientific, Inc., Shoreline, WA (US)

(72) Inventors: Suman Kumar Mulumudi, Snohomish, WA (US); Daniel J. Blase, Everett, WA (US)

(21) Appl. No.: 17/232,091

(22) Filed: Apr. 15, 2021

Related U.S. Application Data
(60) Provisional application No. 63/012,028, filed on Apr. 17, 2020.

(51) Int. Cl. A61B 7/04; H04R 1/46

(52) U.S. Cl. CPC A61B 7/04; H04R 1/46

(57) ABSTRACT

An electronic acoustic collector is disclosed wherein a sealed column of air is created between a diaphragm and an electronic sound sensor. As applied to a stethoscope chestpiece, the device improves audio quality by conducting sound through the sealed column before it reaches the sound sensor.

This application claims priority to U.S. Provisional Application No. 63/012,028, filed Apr. 17, 2020, which is incorporated by reference herein.

### BACKGROUND

Biological acoustic signals are important signs for the identification and treatment of clinical conditions. Collecting biological acoustic signals is challenging given the frequency range at which the signals of interest—such as cardiac and pulmonary sounds—exist. Moving beyond traditional mechanical stethoscopes, various electronic devices have been conceived in recent years in an attempt to collect and analyze these biological sounds using equipment that is portable, compact, and able to transmit and receive electronic signals. However, there are notable drawbacks to many of these devices, such as bulky size, short battery life, high costs of manufacture, and poor acoustic collection capabilities.

A solution is thus needed for an electronic stethoscope that is compact, relatively inexpensive to manufacture, has ease of use, and is capable of high quality sound collection. By combining such a device with software that enables biological sounds to be recorded, analyzed, and transmitted, and ideal tool is presented for internet-based medical diagnostics (often referred to as “telemedicine”).

The tool presented herein could also be used to listen to other kinds of sounds that may be picked up from placing the device in contact with a surface, such as mechanical equipment sounds, sounds reverberating through buildings or machinery, or underwater sounds.

### BRIEF DESCRIPTION OF THE DRAWINGS

FIG. 1 is a cross-sectional view of an embodiment of the devices disclosed herein.

FIG. 2 is an exploded view of the device shown in FIG. 1.

FIG. 3 is a cross-sectional view of an embodiment of the devices disclosed herein.

### DETAILED DESCRIPTION

Devices and methods of construction are disclosed herein for an electronic stethoscope comprising a chestpiece that generally mimics the structure and appearance of a traditional non-electronic stethoscope chestpiece, but with a unique arrangement of mechanical and electronic parts housed in its interior.

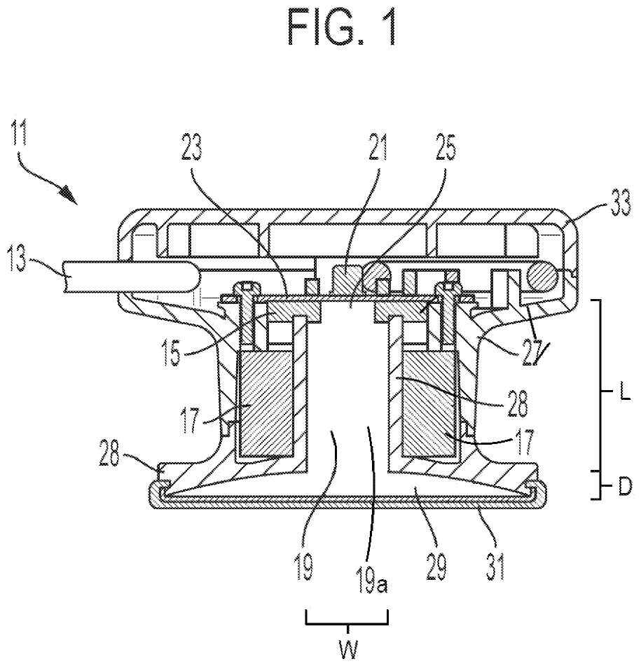

With reference to FIG. 1, a powered electronic chestpiece 11 is shown in a side cross-sectional view. The dimensions of the embodiment of the chestpiece 11 shown in FIG. 1 are roughly that of a traditional non-electronic chestpiece, with a maximum diameter of about 55 mm, and a height of about 36 mm, and such dimensions have been shown effective in collecting human heart and lung sounds. Other sizes and proportions are possible, and may be chosen to best suit the needs of a particular application.

The chestpiece 11 has an external housing that includes a cap 33 and a side housing 27. The chestpiece 11 also has a channel member 28 (also referred to herein as a “bell”) composed of a body or structure that has a central hollow channel 19 and a hollow parabolic cavity 29. While a parabolic cavity may often be preferred for acoustic collection, the device can function if the walls of cavity 29 are merely flat or have some other contour rather than parabolic, and so the walls of cavity 29 may also be referred to herein simply as a flared projection extending concentrically away from the channel 19 so as to form a cavity adjacent to the channel 19. One end of the chestpiece 11 has a diaphragm membrane 31 much like a traditional stethoscope. The hollow parabolic cavity 29 within the bell 28 of the chestpiece 11 is covered by the diaphragm 31, which may, for example, be constructed of a thin piece of plastic capable of reverberating to transmit the desired sound frequencies. The parabolic cavity 29 leads to the hollow channel 19 running axially through the center of the bell 28 of the chestpiece 11 for transmitting sound waves through the chestpiece 11. The external housing 27 of the chestpiece 11 creates a seal around a sleeve 17 and the hollow channel 19.

With respect to dimensions, for use of the device as a traditional stethoscope, for example, use can be made of the device wherein the ratio of the length L of the channel 19 (wherein length is measured from the opening to the cavity 29 to the electronic sound sensor 21) to the depth D of the cavity 29 (wherein depth is measured from the plane of the outer opening of the cavity to the adjacent entrance of the channel) ranges from 5:1 to 20:1. Other ratios are also useable, though may have poorer sound quality for traditional stethoscope uses. Likewise, certain embodiments used as traditional stethoscopes can have a ratio of the inner width (or diameter) of the channel 19 to the width (or diameter) of the maximum diameter of cavity 29 ranging from 1:2 to 1:10. Furthermore, certain embodiments used as traditional stethoscopes with generally cylindrical channels 19 can have a ratio of the length of the channel 19 to its maximum inner width W (or diameter) in the range of 1:1 to 4:1. Again, other ratios are possible, though may have poorer sound quality for traditional stethoscope uses.

The hollow channel 19 is filled with a column of acoustically conductive medium 19a. The medium 19a may simply be air, or in some embodiments may comprise a solid material, a semi-solid material, a liquid, a gelatinous material, or a combination thereof. The selection of the medium 19a will depend on the kind of acoustics desired to be collected, which may vary depending on what is being listened to, and the kind of microphone employed in the device. The selection of the medium 19a can also be used to separate frequencies of interest from the unwanted frequencies.

While shown in FIG. 1 as having a generally cylindrical shape, the hollow channel 19 might also be designed to have a non-cylindrical shape, tapering to a greater degree in width, and/or having flat rather than rounded walls. Modifying the shape of the hollow channel 19 will help attenuate unwanted frequencies and transmit frequencies of interest. Similar attenuation of sound frequencies can be accomplished by changing the length of the hollow channel 19.

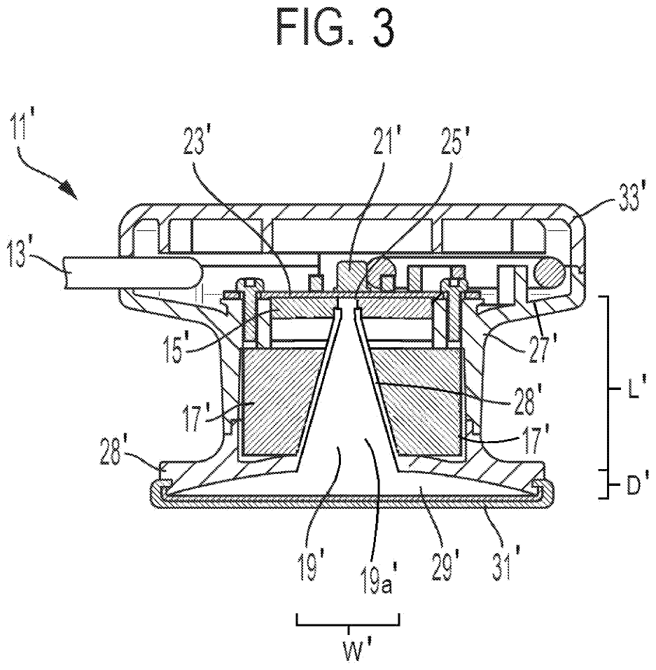

For example, as FIG. 3 depicts a cross-sectional view of a conical hollow channel 19′. The numerals in FIG. 3 using the (prime superscript correspond to their counterparts in FIG. 1. As used herein, “conical” also encompasses a parabolic shape. The narrow end of the conical channel 19′ is located at the electronic sound sensor 21′ (described further below), and has an opening therein to allow transmission of sound to the sound sensor 21′. In certain embodiments in which the cross-sectional shape of the bell is conical, the ratio of the length L′ of the channel 19′ to its maximum width W′ can be in the range of 3:1 to 1:4. Other ratios are possible, though may have poorer sound quality for traditional stethoscope uses. In conical embodiments, use can be made of the device wherein the ratio of the length L′ of the channel 19′ (wherein length is measured from the opening to the cavity 29′ to the electronic sound sensor 21′) to the depth D′ of the cavity 29′ (wherein depth is measured from the plane of the outer opening of the cavity to the adjacent entrance of the channel) ranges from 5:1 to 20:1. In certain embodiments, the bell might not have a separate flared projection or cavity 29′, with the flared projection simply comprising the end of the channel 19′ itself

When capturing low frequency sounds like those from a human heart and lungs, it is generally advantageous to utilize the hollow channel 19 to allow the sound waves to propagate. If the propagation area is too small, the sound waves will be muffled; if too large, the sound waves may dissipate.

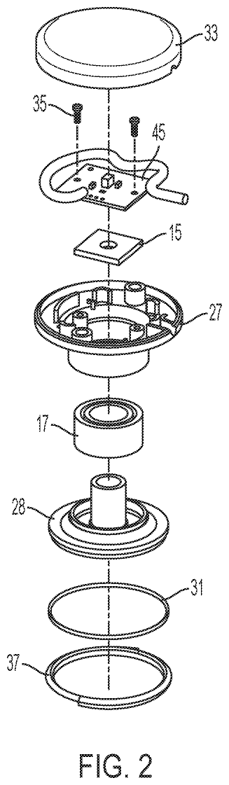

For purposes of listening to human heart and lung sounds, a hollow channel 19 of the general proportions shown in FIGS. 1 and 2 has been shown to work satisfactorily when filled simply with air at ambient pressure. Also, it has been shown that constructing the bell 28 out of rigid plastic will adequately reflect human heart and lung sounds. Other material types can be used for the bell, and generally speaking the stiffer the material, the better it will reflect sound (though stiff materials may require more insulation around them to minimize interference from external vibration sources). Other variations of size and shape may be utilized for the purpose of listening to heart and lung sounds so long as the combination of material properties and volume allow sufficient propagation of the desired frequencies.

A toroidal or doughnut-shaped sleeve 17 encircles the bell 28 and hollow channel 19. The sleeve 17 can serve two general purposes: (1) to add mass to the device so that it has more stability when held by a human user and when pressed against a listening surface, and (2) to act as a sound and vibration dampener to minimize sound and other vibrations that might be transmitted to the hollow channel 19 from the exterior of the device through its housing. In this second respect, the sleeve 17 is acting to help isolate the hollow channel 19 from its surrounding environment such that the primary vibrations that it receives are those propagated by the diaphragm. Alternatively or in addition to (2), the sleeve 17 can be made to reinforce the walls of the hollow channel 19 to improve their ability to reflect sound.

Increasing the mass of the sleeve 17 will assist with objective (1) of giving the chestpiece 11 more stability when held by a human user. In the embodiment shown in FIGS. 1 and 2, the sleeve 17 contains metal for this purpose. Metal can also be incorporated into the housing of the chestpiece 11 for this same purpose, though it has been shown that a rigid plastic housing will allow collection of human heart and lung sounds. In certain embodiments, the sleeve can be designed so as to account for the majority of the mass of the entire chestpiece unit so as to impart substantial weight.

If the sleeve comprises (in whole or in part) a hollow toroidal shell containing vibration dampening material such as, for example, foam or gel, this will assist in minimizing interference from vibrations transmitted through the housing of the chestpiece 11 from external sources. Vibration dampening material (such as foam or gel) can otherwise be used in the fittings among and between parts of the chestpiece housing to achieve this same purpose. In some embodiments, most or all of the negative space of the chestpiece 11 surrounding the bell 28 can be filled with a foam or gel, which can also replace the space otherwise occupied by the sleeve.

At the end of the hollow channel 19 opposite from the diaphragm is a gasket 15 separating the channel member 28 (and the hollow channel 19) from a circuit board (or other mounting structure) 23 mounting an electronic sound sensor 21, such a microphone, piezoelectric crystal, or an ultrasonic motor. In the embodiment shown in FIGS. 1 and 2, a microelectro-mechanical systems (MEMS) microphone is depicted. It has been shown, for example, that a TDK InvenSence ICS-40300 model microphone will work well for listening to human heart and lung sounds in the disclosed device, though other kinds of microphones with similar kinds of functional advantages may be used.

The gasket 15 serves the function of creating a seal between the circuit board 23 and the hollow channel 19 to keep sound waves within the hollow channel 19. The gasket 15 can be made to have a toroid or doughnut shape with a hollow center channel 25 under the sound sensor 21 to allow direct interaction with the sound waves propagating in the hollow channel 19.

The choice of the structure and material of the gasket 15 will affect the properties of the sound reaching the sensor, and may be varied depending on the type of sound sensor used, and the acoustic properties of the rest of the device. In the embodiment shown in FIGS. 1 and 2, a foam gasket is employed, and has been shown to be effective in sealing the hollow channel 19 and allowing good propagation of air waves.

It is advantageous to construct the gasket 15 (and the other materials contacting the circuit board 23) out of a material that will not generate static, which may damage or overload the circuit board 23. It has been shown that ethylene-vinyl acetate (EVA) foam works well as the material for the gasket, though other materials offering the same or similar properties can be employed for anti-static sealing.

In certain embodiments, it may be advantageous for the gasket 15 to have a visible cut in it that allows air to freely move out of its perimeter and into the rest of the housing of the chestpiece, thereby eliminating the seal around the air column in the channel 19. This might be desirable in instances where the sound reverberation and air pressure in the hollow channel 19 is such that it might otherwise overwhelm the microphone. For purposes of listening to human heart and lung sounds using the embodiment shown in the drawings, no such venting was needed, and a full seal was found to work well for ensuring good audio quality.

Certain examples described herein can provide advantages relating to the positioning of the sound sensor away from the diaphragm at the opposite end of a hollow channel. Certain other electronic stethoscopes position a microphone adjacent to the diaphragm, which can result in poorer sound quality. The device disclosed herein leverages the acoustic attenuation of the hollow channel to faithfully reproduce sounds received by the sensor. The cavity created by the hollow channel acts to direct sound waves to the microphone and results in better sound reproduction at the microphone at the other end of the channel, not unlike how sound may propagate within an ear canal before hitting the diaphragm.

Furthermore, in the embodiment shown in FIGS. 1 and 2, the system comprises a sealed column of air with a diaphragm at one end and a microphone at the other, wherein the design of the device generally minimizes external vibrations from acting on that system other than the sound waves conducted into the system by the diaphragm. As used here, the term “sealed” can include air-tight embodiments, as well as embodiments in which some nominal air leakage may occur while nonetheless still maintaining a substantially isolated column of air.

The sound sensor 21 is electrically connected via an electrically wired connection 13 to an external powered electronic device (not shown), such as a computer or a smartphone. By employing a smartphone, the device disclosed herein can be easily used for telemedicine purposes. The electronic device provides power to the sound sensor 21, thereby eliminating the need to have a battery within the chestpiece 11, which increases bulk, expense, inconvenience, and hinders sound quality. Wired connection 13 may also carry electronic audio signals.

The electronic device may be capable of receiving and processing electronic signals from the sound sensor 21, and either analyzing those signals itself, or transmitting them to another device that can. When paired with a software program, the electronic signals from the sound sensor 21 can be recorded, played back as audio over speakers, and analyzed to detect certain target sound signatures or anomalies, thereby acting as a diagnostic tool.

In alternative embodiments, an on-board power source and computer may be used, though these would increase the bulk, weight, and expense of the device.

Referring now to FIG. 2, an exploded perspective view is shown of chestpiece 11 from FIG. 1. Element 45 is the unit comprising wired connection 13, circuit board 23, and sound sensor 21. Screws 35 connect the circuit board 23 to the side housing 27, and apply pressure to the gasket 15 to ensure a good seal. A ring 37 seals the diaphragm 31 about the end of the bell 28. Note that the bell 28 contacts the diaphragm end of the chestpiece 11, but the walls of the hollow channel 19 are otherwise relatively isolated from the rest of the device housing, and the hollow channel 19 is sealed at the other end by the gasket 15, all of which helps minimize interference from external vibration, and ensure that the primary vibrations in the hollow channel 19 are those derived from the diaphragm 31. The cap 33 forms a tight fit over the chestpiece 11 to minimize exterior elements acting on the sound sensor. The cap 33 can be friction fitted to the rest of the chestpiece 11, and/or adhered with adhesive or welds, or other suitable fastening mechanism.

In examples described herein, the external housing of the device, as well as the bell 28, may be made of any suitable material or materials including, but not limited to metal, plastic, ceramic, composite material, or combinations thereof. Hard plastic has been shown to provide acceptable sound quality.

Although various particular embodiments of the invention have been illustrated and described herein, it will be appreciated that other embodiments, and equivalences thereof, can be made and that many changes can be made without departing from the principles of the invention. Furthermore, the embodiments described above can be combined to form yet other embodiments of the invention of this application. Accordingly, it is to be distinctly understood that the foregoing descriptive matter is to be interpreted nearly as illustrative of the invention and not as a limitation.

What is claimed is:

1. An electronic stethoscope chestpiece comprising:
(a) a bell comprising:
(i) a cylindrical hollow channel with a first end and a second end; said channel having a length to inner diameter ratio in the range of 1:1 to 4:1;
(ii) a flared projection extending concentrically outward from said second end of said channel and forming a hollow cavity adjacent to said second end of said channel, said flared projection having an open end;
(b) a diaphragm covering said open end of said flared projection;
(c) a mounting structure covering said first end of said channel, and said mounting structure holding an electronic sound sensor at said first end of said channel.

2. The chestpiece of claim 1 wherein said bell contains an air column, and wherein said air column is sealed within said bell by said mounting structure at said first end of said channel, and said diaphragm at said open end of said flared projection.

3. The chestpiece of claim 2 including a gasket between said bell and said mounting structure.

4. The chestpiece of claim 2 wherein said seal is air tight.

5. The chestpiece of claim 1 including an external housing, and a sleeve within said external housing fitting around said bell, wherein said sleeve comprises the majority of the mass of said chestpiece.

6. The chestpiece of claim 1 including an external housing, and a sleeve within said external housing fitting around said bell, wherein said sleeve is composed of a metal.

7. The chestpiece of claim 1 including an external housing, and sound dampening foam located in at least a portion of the region between the inside of said external housing and said bell.

8. The chestpiece of claim 1 wherein the ratio of the length of said channel to the depth of said cavity ranges from 5:1 to 20:1.

9. The chestpiece of claim 8 wherein said bell contains an air column, and wherein said air column is sealed within said bell by said mounting structure at said first end of said channel, and said diaphragm at said open end of said flared projection.

10. The chestpiece of claim 8 including a gasket between said bell and said mounting structure.

11. The chestpiece of claim 8 wherein said seal is air tight.

12. The chestpiece of claim 1 wherein said electronic sound sensor has a wired connection leading away from said chestpiece, and wherein another end of said wired connection is connected to an external power source capable of powering said electronic sound sensor.

13. The chestpiece of claim 12 wherein said bell contains an air column, and wherein said air column is sealed within said bell by said mounting structure at said first end of said channel, and said diaphragm at said open end of said flared projection.

14. The chestpiece of claim 13 including a gasket between said bell and said mounting structure.

15. The chestpiece of claim 13 wherein said seal is air tight.

16. An electronic stethoscope chestpiece comprising:
(a) a bell comprising a hollow channel with a first end and a second end; said channel having conical cross-sectional shape; the narrow end of said channel being located at said first end and having an opening therein to allow transmission of sound to an electronic sound sensor; said second end of said channel terminating in a flared projection with an open end; said flared projection selected from the group consisting of (i) said second end of said channel and (ii) a further projection not part of said channel having a greater width than said second end of said channel with said further projection forming a cavity adjacent to said second end of said channel; the ratio of the length of said channel to its width at said second end being in the range of 3:1 to 1:4;
(b) a diaphragm covering said open end of said flared projection;
(c) a mounting structure covering said opening of said first end of said channel, and said mounting structure holding an electronic sound sensor at said first end of said channel.

17. The chestpiece of claim 16 wherein said flared projection is a further projection not part of said channel having a greater width than said second end of said channel, and wherein the ratio of the length of said channel to the depth of said cavity ranges from 5:1 to 20:1.

18. The chestpiece of claim 16 wherein said bell contains an air column, and wherein said air column is sealed within said bell by said mounting structure at said first end of said channel, and said diaphragm at said open end of said flared projection.

19. The chestpiece of claim 18 including a gasket between said bell and said mounting structure.

20. The chestpiece of claim 18 wherein said seal is air tight.
